(19) United States
(12) Patent Application Publication
(10) Pub. No.: US 2013/0203069 A1
(43) Pub. Date: Aug. 8, 2013

(54) Dynamic Assay for Maintenance and Disruption of Tissue Level Organization and Architecture

(71) Applicant: The Regents of the University of California, Oakland, CA (US)

(72) Inventors: Mark A. LaBarge, Orinda, CA (US); Mina J. Bissell, Berkeley, CA (US); Martha R. Stampfer, Oakland, CA (US)

(73) Assignee: THE REGENTS OF THE UNIVERSITY OF CALIFORNIA, Oakland, CA (US)

(21) Appl. No.: 13/762,309

(22) Filed: Feb. 7, 2013

Related U.S. Application Data
(60) Provisional application No. 61/596,178, filed on Feb. 7, 2012.

(51) Int. Cl. C12Q 1/02

(52) U.S. Cl. CPC C12Q 1/02
USPC 435/7.1; 435/29

(57) ABSTRACT

A micropatterning approach, systems and methods that confine cells to a specified geometry combined with an algorithm to quantify changes of cellular distribution over time to measure the ability of different cell types to self-organize relative to each other and detect loss of cellular self-organization.

### CROSS-REFERENCE TO RELATED APPLICATIONS

This application is a non-provisional application of and claiming priority to U.S. Provisional Patent Application No. 61/596,178 filed on Feb. 7, 2012, and hereby incorporated by reference in its entirety.

### STATEMENT OF GOVERNMENTAL SUPPORT

This invention was made with government support under Contract No. DE-AC02-05CH11231 awarded by the U.S. Department of Energy, under Grant No. R00AG033176 from the National Institute on Aging, Grant U54CA112970 from the National Cancer Institute, and by Laboratory Directed Research and Development (LDRD) funding from the Lawrence Berkeley National Laboratory, provided by the Director, Office of Science, of the US Department of Energy under Contract DE-AC02-05CH11231, by National Cancer Institute Grants R37CA064786, U54CA126552, R01CA057621, U54CA112970, U54CA143836, and U01CA143233; and by US Department of Defense Grants W81XWH0810736, BCRP BC060444 and U54CA112970. The government has certain rights in the invention.

### BACKGROUND OF THE INVENTION

1. Field of the Invention

The present invention relates to assays for determining cellular organization in tissues and the effect of various factors on cellular organization or disruption in tissues.

2. Related Art

Most mammalian adult tissues are replenished and repaired throughout life by reservoirs of stem cells. As new somatic cells replace old ones or build new tissue, organization and architecture must be maintained. The alternative, loss of organization in adult tissues, is associated with cancer and other diseases. Lineage-specific progenitors or their differentiated progeny must have a means to reach their ultimate site of residence within the adult tissue. The robust ability to organize cells into tissues is marked from conception: Heterogeneous aggregates of dissociated cells from embryonic tissues, suspended in gels or hanging droplets or on agarose-coated plates, self-organize into semblances of the original tissues (See Steinberg M S (1962) Mechanism of tissue reconstruction by dissociated cells. II. Time-course of events. Science 137:762-763; (1962) On the mechanism of tissue reconstruction by dissociated cells, Iii. Free energy relations and the reorganization of fused, heteronomic tissue fragments. Proc Natl Acad Sci USA 48:1769-1776 and Townes P L, Holtfreter J (1955) Directed movements and selective adhesion of embryonic amphibian cells. J Exp Zool 128:53-120, and Wei C, Larsen M, Hoffman M P, Yamada K M (2007) Self-organization and branching morphogenesis of primary salivary epithelial cells. Tissue Eng 13:721-735)).

The mechanisms governing self-organization during developmental morphogenesis (Foty R A, Steinberg M, (2004) Cadherin-mediated cell-cell adhesion and tissue segregation in relation to malignancy. Int J Dev Biol 48:397-409; Foty R A and Steinberg M, (2005) The differential adhesion hypothesis: A direct evaluation. Dev Biol 278:255-263; Krieg M, et al., (2008) Tensile forces govern germ-layer organization in zebrafish. Nat Cell Biol 10:429-436; Shi Q, Chien Y H, Leckband D, (2008) Biophysical properties of cadherin bonds do not predict cell sorting. J Biol Chem 283:28454-28463; Manning M L, Foty R A, Steinberg M S, Schoetz E M (2010) Coaction of intercellular adhesion and cortical tension specifies tissue surface tension. Proc Natl Acad Sci USA 107(28):12517-12522) are likely conserved in the maintenance of organization in adult tissues.

For example, the mammary gland undergoes cycles of proliferation and involution, showing as much as a 10-fold expansion in preparation for lactation followed by return to normal size. During these processes, the precise bilayered branching organization throughout the gland is maintained; secretory luminal epithelial cells (LEPs) line the lumen, surrounded by a layer of contractile myoepithelial cells (MEPs) that are adjacent to the basement membrane. Understanding cellular capabilities to self-organize would help explain how, for instance, the mammary stem cell-enriched zone in the ducts (Villadsen R, et al. (2007) Evidence for a stem cell hierarchy in the adult human breast. J Cell Biol 177:87-101) is maintained separately from the rank-and-file LEPs and MEPs, and how LEPs and MEPs form and maintain bilayers.

The self-organization process that drives heterogeneous mixtures of cells to form organized tissues is well studied in embryology and with mammalian cell lines that were abnormal or engineered. However, the phenomenon of self-organization has not been well studied in humans, perhaps because of the challenges of working with primary materials and a paucity of tractable culture systems for maintaining cell types from normal adult tissues.

Previous methods used to study self-organizing behavior of cells in tissue were not quantifiable, thereby preventing dynamic longitudinal studies. In previous work randomly formed aggregates of cells were cultured atop of agarose, in hanging drops or in spinning culture flasks which did not permit observation over long periods of time, inability to detect small changes in phenotype, and observation of proper organization only occurs inconsistently in certain aggregates of certain sizes and shapes. Thus, there is a need for a robust assay that is capable of facilitating screens for specific activities for extended periods of time.

### BRIEF SUMMARY OF THE INVENTION

The present invention provides for methods to quantify changes in the distributions of cells that make up tissues, such as epithelial tissue, to distinguish normal cell organization from abnormal organization.

Loss of organization is a principle feature of cancers; therefore it is important to understand how normal adult multilineage tissues, such as bilayered secretory epithelia, establish and maintain their architectures. Herein in one embodiment is described a micropatterning approach that confines cells to a cylindrical geometry combined with an algorithm and method to quantify changes of cellular distribution over time to measure the ability of different cell types to self-organize relative to each other.

Thus in some embodiments, a method for determining loss of organization in a tissue, comprising the steps of: (a) providing a microwell substrate; (b) providing labeled cells from said tissue in the microwells; (c) allowing said cells to self-organize into a specified geometry for a period of time; (d) detecting the changes in localization of said cells over time to self-organize into a specified geometry, whereby little to no changes in localization of said cells over time indicates a loss of organization in said tissue.

In some embodiments, the label is an antibody stain, fluorescence, or a membrane staining fluorescent dye.

In other embodiments, the microwell substrate may be comprised of a glass or polymer composition. In various embodiments, the polymer selected from the group consisting of polydimethylsiloxane, polyethyleneglycol, polyacrylamide, polyacrylamide conjugated to collagen 1, and agarose.

In various embodiments, the method further comprising pre-treating the cells with a test or environmental factor, wherein the pre-treatment is a nucleic acid, peptide, protein, drug, small molecule, inhibitor, analyte, chemical, virus, mutation, radiation, temperature change, transformation, or media.

In other embodiments, the cells may be a heterogeneous mixture of cell types, wherein each cell type is labeled differently. In some embodiments, the cells are a mixture of cell of normal human epithelial cells, mixtures of normal and transformed human epithelial cells, or combinations of embryonic or induced pluripotent state stem cells with normal epithelial cells.

In various embodiments, the tissue is any pseudostratified epithelial tissue including but not limited to breast, prostate, ovarian, testes, skin, internal organ, etc.

In various embodiments, the detection of the changes in localization of the cells over time further comprises imaging the labeled cells over time and analyzing the images of the labeled cells. Analyzing the images of the labeled cells in some embodiments is comprised of the steps of (i) calculating the mean intensity values of each label for every pixel along the radius or outside edge of the images of the labeled cells, and (ii) determining the log₂ ratio of the mean intensity values, wherein the greater the distance of the log₂ ratio from the center line indicates changes in the localization of the labeled cells and self-organization is detected. In other embodiments, if the log₂ ratio that stays close to the center line indicates no self-organization was detected and a loss of organization in the tissue.

Therefore, in other embodiments, the present invention also provides a computer-implemented process comprising the steps of (1) imaging the labeled cells over time and (2) analyzing the images of said labeled cells, wherein the analyzing step (2) of the images of said labeled cells comprised of the steps of (i) calculating the mean intensity values of each label for every pixel along the outside edge of the images of said labeled cells, and (ii) determining the log₂ ratio of the mean intensity values, wherein the greater the distance of the log₂ ratio from the center line indicates changes in the localization of said cells and self-organization is detection, and wherein the log₂ ratio that stays close to the center line indicates no self-organization was detected and a loss of organization in said tissue. In another embodiment, a system carrying out the computer-implemented process.

### BRIEF DESCRIPTION OF THE DRAWINGS

FIG. 1 shows that heterogeneous mixtures of luminal and myoepithelial cells self-organize into ordered structures. (A) Flow cytometry analysis of fourth-passage finite-lifespan HMEC strain 240L reveals distinct populations of the two principal somatic epithelial lineages of mammary gland: MEPs, defined here as CD227⁻/CD10⁺, and LEPs, defined as CD227⁺/CD10⁻. (A′ and A″) Immunofluorescence of sorted cells for MEP and LEP markers K14 (red) and K19 (green), respectively, verified that (A′) CD227⁺ LEPs were K14⁻/K19⁺, and (A″) CD10⁺ MEPs were K14⁺/K19⁻. Nuclei were counterstained with DAPI (blue). (B) Images of mixtures of fluorescently labeled LEPs (green) and MEPs (red) suspended in hanging droplets and imaged with a confocal microscope just after mixing at 0 h (Left) and at 48 h (Right). (Scale bars: 20 μm.) (C) Cartoon representation of the microwell self-organization assay. Fluorescently labeled LEPs (green) and MEPs (red) were mixed together and placed in arrays of microwells that did not support cell adhesion. Thirty wells were imaged with a confocal microscope just after the addition of cells (0 h) and again at 24 h and 48 h. Fluorescence from both green and red channels in one optical section per well was binarized and then combined and averaged to generate two gray-scale composite images that were overlaid to generate a single two-color composite distribution map for each condition, with LEP distributions in green and MEP distributions in red. (D) Representative fluorescence images of LEP (green) and MEP (red) in four different microwells at 0 h and 48 h (Upper) and of controls, which were heterogeneous HMEC arbitrarily labeled with red or green fluorescent labels (Lower). (E) Distribution maps of LEP (green) and MEP (red) (Upper) or control mixtures at the 0-h, 24-h, and 48-h time points (Lower). (E′) Quantification of heat maps in E showing changes in mean distribution of red and green pixels along the radius around 360° of arc at three time points. Green lines show 0 h, red lines show 24 h, and blue lines show 48 h. SD is shown by the lightly shaded regions of colors corresponding to each line.

FIG. 2 shows that epithelial lineages that comprise the mammary gland express E-cadherin differentially. (A) A tissue section from a normal mammary gland, specimen XD01, embedded in paraffin and triple-immunostained to show expression of (Left) the MEP and LEP markers K14 (red) and K19 (green), respectively, and (Right) E-cadherin (gray scale). (B) Dot plots show image quantification from two individuals, XD01 and XA01, of E-cadherin protein levels at the border between two LEPs (K19+ LEP) and at the border between an LEP and an MEP cell (K14+ LEP) and background fluorescence as measured on stroma, which does not express E-cadherin (Stroma). Measurements are expressed in arbitrary fluorescence units (afu), n=100 for each cell type collected from at least three sections. (C) Flow cytometry analysis of E-cadherin expression in CD10⁺ MEPs and CD227⁺ LEPs in HMEC strains at fourth or fifth passage from six individuals.

FIG. 3 shows that E-cadherin-containing junctions and the cytoskeleton regulatory molecules ROCK and MLCK are required for self-organizing. (A) Maps of HMEC lineage distributions over time in the presence of E-, P-, or VE-cadherin-blocking agents (anti-E-cadherin, recombinant (rec) E-cadherin, anti-P-cadherin, or recVE-cadherin); LEP are green, and MEP are red. (B) LEP (green) and MEP (red) distributions in the presence of the MLCK inhibitor ML-7 or the ROCK inhibitor Y27632. (A′ and B′) Quantification of heat maps in A and B, respectively, showing changes in mean distribution of red (MEP) and green (LEP) pixels along the radius around 360° of arc at three time points. Green lines show 0 h, red lines show 24 h, and blue lines show 48 h. SD is shown by the lightly shaded region of color corresponding to each line. (C) Atomic force microscopy measurements of LEP and MEP in the presence of ML-7 and Y27632. The graph represents elasticity (kPa) values for 45 cells per condition; the interior line represents mean elasticity values; error bars show SE. Strain 240L at fourth passage was used for all experiments.

FIG. 4 shows Self-organization among luminal and myoepithelial cells is driven by E-cadherin activity. (A) E-cadherin protein expression at the surface of LEPs and MEPs in the presence of ML-7 or Y27632, as measured by flow cytometry. (B) Flow cytometry analysis of recombinant E-cadherin (rEcad) binding to HMEC cell surfaces. (C) LEP (green) and MEP (red) distribution maps showing the impact on self-organization over time (time points: 0 h, 24 h, and 48 h) when the inhibitors ML7 and Y27632 were added at 0 h or after 24 h. (C) Quantification of heat maps in C showing changes in mean distribution of red (MEP) and green (LEP) pixels along the radius around 360° of arc at three time points: green lines show 0 h, red lines show 24 h, and blue lines show 48 h. SD is shown by the lightly shaded region of color corresponding to each line. Strain 240L at fourth passage was used for all experiments.

FIG. 5 shows a single Frame from a time-lapse movie of HMEC self-organization. LEP (green) and MEP (red) enriched from strain 240LB were time-lapse imaged in a microwell every 30 min from +6 h to +48 h.

FIG. 6 shows a graphical explanation of a method for quantifying cellular distributions in heat maps.

### DETAILED DESCRIPTION OF THE PREFERRED EMBODIMENT

### Introduction

To facilitate a quantitative understanding of how cells self-organize into domains of lineage specificity and tissue organization processes in an adult epithelial tissue, an assay and systems were developed. In some embodiments, as describe herein an assay to identify and track cellular self-organizing behavior. In some embodiments, a robust cell culture system is provided that enables culture of normal multiple cell (sub)types for multiple population doublings and enrichment of cells from multiple lineages, followed by the cells being placed in arrays of micropatterned microwells where their distributions and localizations are tracked over time to generate a dynamic understanding of lineage-specific self-organizing behavior.

In one embodiment, the cell culture system enables studies of self-organization of LEP and MEP lineages that comprise cultured pre-stasis normal HMEC strains derived from reduction mammoplasties. Flow cytometry-enriched cells from both lineages were placed in arrays of micropatterned microwells, where the distributions of both lineages were imaged and tracked over time.

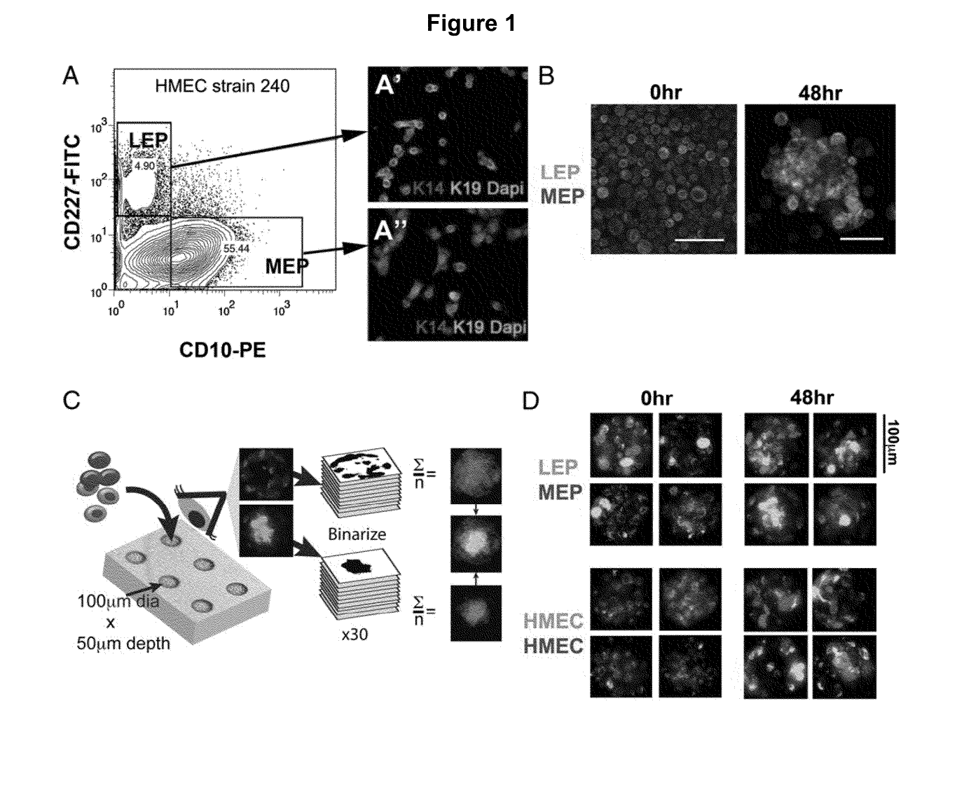

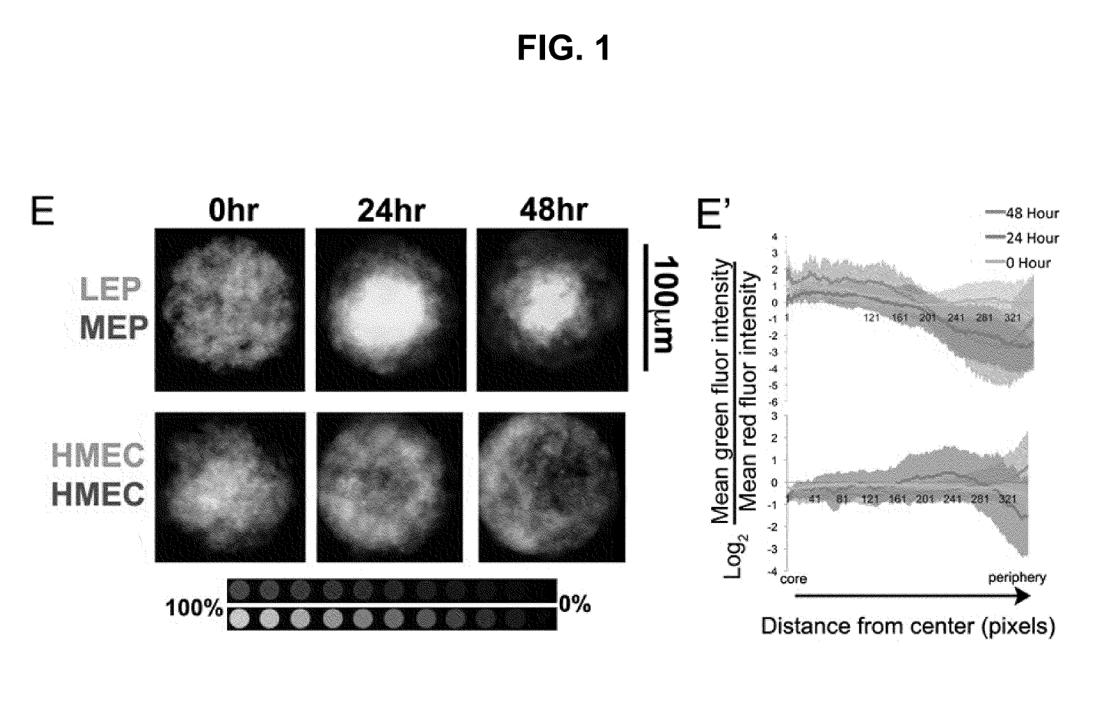

Thus, in one embodiment, a microwell self-organization assay is described that confines cells to a cylindrical geometry combined with an algorithm and method to quantify changes of cellular distribution over time to measure the ability of different cell types to self-organize relative to each other. A microwell culture platform can be engineered that confines the cell mixtures whereby the cells can organize to adopt a specified geometry, which enables quantification of lineage distributions over time. In various embodiments, the specified geometry detected is a three-dimensional (3D) cylindrical geometry. For example, the myoepithelial and luminal HMEC cells self-organize into such a 3D cylindrical geometry (See. FIG. 1E) with the luminal cells distributed inside the outer cylinder of myoepithelial cells as they do in vivo.

Any suitable mode of imaging or detection of the 3D geometry may be used. In some embodiments, representative optical sections of heterogeneous labeled cell mixtures in the microwells, taken at middepth (e.g., ˜25 μm) at various timepoints, should allow determination of whether self-organization has occurred, as compared with mixtures of arbitrarily labeled cell cultures.

In some embodiments, micropatterned substrata can be made according to Tan et al. ((2004) Simple approach to micropattern cells on common culture substrates by tuning substrate wettability. Tissue Eng 10:865-872), hereby incorporated by reference. Briefly, the method is used to micropattern cells onto a solid substrate such as glass or a polymer. In one embodiment, an elastomeric polymeric stamp is used to contact-transfer extracellular matrix protein onto a surface followed by blocking cell adhesion in the surrounding regions by the physisorption of Pluronic surfactants. In some cases, adjusting the wettability of the tissue culture substrate allows the micropatterning of cells.

In one embodiment, polymer microwell arrays are formed by curing prepolymer against a prepatterned master. The arrays of wells are peeled away and cut into pieces that are affixed with a few microliters of uncured polymer to the bottom of a multi-well plate. Plates with microwells are UV oxidized, blocked with a protein such as BSA under vacuum, and rinsed appropriately such as with PBS and media. In various embodiments, various or any polymer or polymer composition can be used to form the microwell arrays. In some embodiments, the polymer is polydimethylsiloxane (PDMS). However as shown in Table 1 below, certain polymers and/or polymer compositions such as agarose or polyethylene glycol (PEG) when used to form the wells may result in different cell organizations. It is likely that proteins cannot adsorb to the surface as readily thus resulting in for example, inversion of the cell organization.

TABLE 1
Examples of Polymer Compositions and Their Effect
Upon HMEC Organization

| | | Resulting Effect |
|---|---|---|
| Polymer | Composition | on HMEC Organization |
| Polydimethylsiloxane | 10:1 (cure:polymer) | Luminal cells inside of |
| | | myoepithelial cells |
| | | (normal) |
| Polyethyleneglycol | multiple | Myoepithelial cells |
| | | inside of luminal cells |
| | | (inverted) |
| Polyacrylamide | 10% acrylamide, 0.3% | Myoepithelial cells |
| | bisacrylamide | inside of luminal cells |
| | | (inverted) |
| Polyacrylamide | 10% acrylamide, 0.3% | Luminal cells inside |
| conjugated to | bisacrylamide | of myoepithelial cells |
| collagen 1 | | (normal) |
| Agarose | 3% | Myoepithelial cells |
| | | inside of luminal cells |
| | | (inverted) |

A heterogeneous cell mixture(s) introduced into arrays of polymer-casted microwells. Different cells and/or cell subtypes can be used to produce heterogeneous cell mixtures. In various embodiments, the cells in the mixtures comprised of at least two different cell types. In some embodiments, the cell mixtures comprised of at least two different cell subtypes found in the same tissue. For example, the Examples and the Figures use mixtures of myoepithelial and luminal subtypes of human mammary epithelial cells. In other embodiments, the cell mixtures comprised of cells from two different subjects, different subjects of different ages, cells having undergone differential treatments, or cells from different origins. For example, two different or a single heterogeneous mixture of cells comprised of myoepithelial and luminal HMEC cells from two different patients of different ages to observe how cells organize differently with respect to age or comorbidities.

Examples of different treatments of cells include but are not limited to exposure to any test factor or environmental test factor such as a nucleic acid, peptide, protein, drug, small molecule, inhibitor, analyte, chemical, virus, mutation, radiation, temperature change, transformation, media, etc. Exposure can be for any length of time or for limited, periodic, intermittent, or prolonged exposure.

In some embodiments, for self-organizing experiments conducted with HMEC, M87A media should be used as M87A media can support normal pre-stasis HMEC for 40-60 population doublings which in some cases is needed for long-range experiments. Cell culture media and methods that may be applicable are described by Garbe J C, Bhattacharya S, Merchant B, Bassett E, Swisshelm K, Feiler H S, Wyrobek A J, Stampfer M R, “Molecular distinctions between stasis and telomere attrition senescence barriers shown by long-term culture of normal human mammary epithelial cells,” Cancer Res. 2009 Oct. 1; 69(19):7557-68, and in WO 2007/115223 and U.S. Patent Pub. No. US-2010-0022000-A1, all of which are hereby incorporated by reference for all purposes. In other embodiments, appropriate cell media which is able to support multiple population doublings of the cells should be used in order to allow long range experiments or screening to be observed using the assay.

In various embodiments, flow cytometry-sorted cells are stained and washed extensively with medium after staining. Any method of labeling the cells can be used, such as antibody staining, expression of fluorescent proteins, membrane staining fluorescent dyes, etc. For example, DiI and DiO dye-stained HMEC cells are mixed to provide a heterogeneous cell mixture of a predefined ratio and resuspended in media.

The test factor, if any, may be added to the cell suspensions just before the cells are introduced into the microwells and are allowed to load for several minutes to an hour or more, e.g., 30-60 min, according to the predetermined exposure times. Excess cells can be washed away, e.g., with medium.

In one embodiment, in the Examples, anti-E-cadherin antibody inhibitors, anti-P-cadherin antibody inhibitors, or actinomyosin network protein inhibitors added to the medium after excess cells were washed away and at every medium change. Examples of such adherens junction inhibitors include but are not limited to, anti-E-cadherin (100 μg/mL clone HECD-1; Invitrogen); anti-E-cadherin (100 μg/mL clone HECD-1; Invitrogen); anti-P-cadherin (100 μg/mL clone NCC-CAD-299; Abcam); recombinant human (rh) E-cadherin-Fc (recEcad, 100 μg/mL; R&D Systems); rhVE-cadherin (100 μg/mL; R&D Systems). Examples of actinomyosin protein network inhibitors include but are not limited to, Y27632 (10⁻⁵ M; Calbiochem); or ML-7 at 3×10⁻⁶ (Calbiochem).

Cells are imaged or detected at start and various timepoints (e.g., 0, 24, and/or 48 h) to measure cell organization and distribution over time. In some embodiments, images of the cells are taken at an intermediate (e.g., ˜25 μm) z axis position of in the wells. Each condition at each time point is binarized using the Threshold function, merged into a Z-stack, and then averaged using ImageJ software (National Institutes of Health) or the like. The Examples below provide various types of analysis that can be performed to determine the lineage-specific differential cell distribution over time. The algorithm embodied in the code provided below allows one to quantify changes of cellular distribution over time to measure the ability of different cell types to self-organize relative to each other. An example of exemplary code and algorithm for use in the present examples and embodiments:

```
clear all;
% Input maps for analysis
inputImageG = imread(′labarge/rECAD/rE,green,0hAVG_0h.tif′);
inputImageR = imread(′labarge/rECAD/rE,red,0hAVG_0h.tif′);
% Normalize each map to highest intensity
ng = im2double(inputImageG) ./ max (max (im2double (inputImageG)));
nr = im2double(inputImageR) ./ max (max (im2double (inputImageR)));
% Convert maps to express the separation of intensities
% Inf or NaN's can be avoided by giving zero values a small value
ng (ng==0) =0.01;
nr (nr==0) =0.01;
nt = log2 (ng./nr);
% Determine center
imSize = length(inputImageG(1,:));
center = round(imSize/2 + 1);
% Replot with respect to the center
% Array of summed values
unwrap = zeros(360, round(imSize/2));
for theta = 0:359
    display([′Extracting column ′ num2str(theta+1) ′ of 360′]);
    % Rotate the image by the desired amount
    rotated = imrotate(nt,theta, ‘bicubic’,‘crop’);
    % Extract the column from the center to the bottom
    unwrap(theta+1, :) = rotated(center:end,center);
end
% Determine average/standard deviations
for (i = 1:round(imSize/2))
    ntavg(i) =mean( unwrap(:,i));
    ntstd(i) =std (unwrap(:,i));
end
    ntavg=ntavg′;
    ntstd=ntstd′;
```

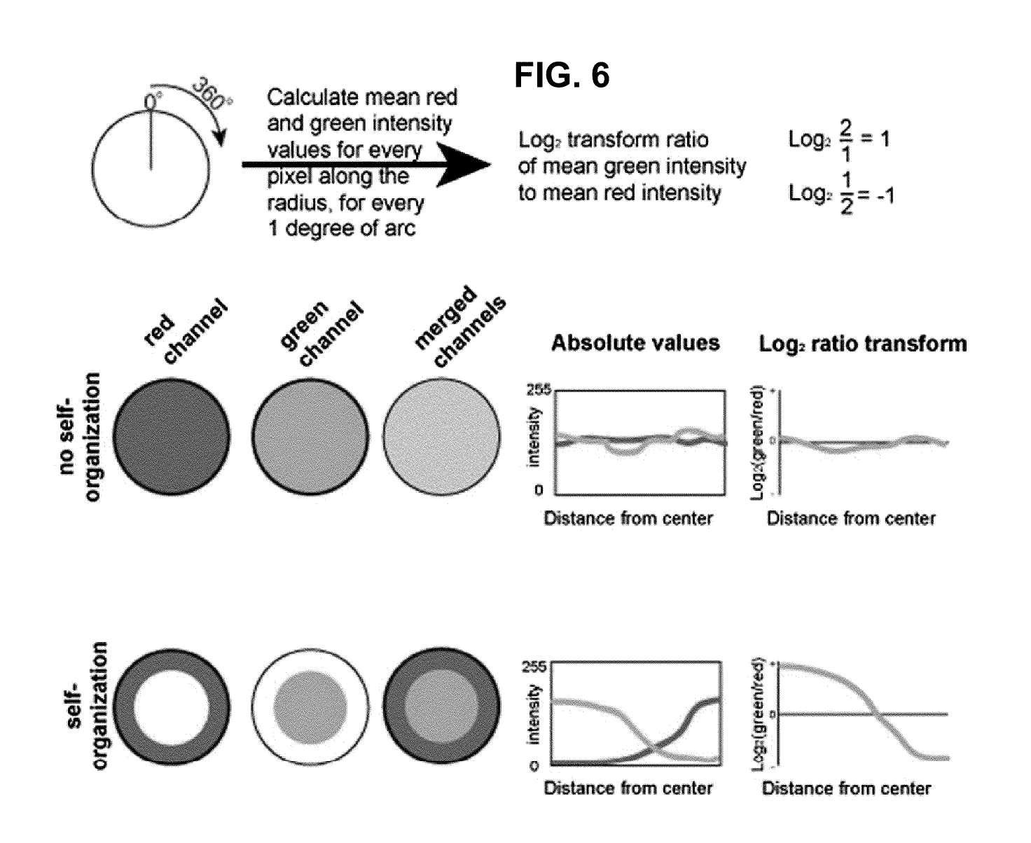

Thus, systems and methods are also provided to measure the ability of different cell types to self-organize relative to each other. In some embodiments, the changes of cellular distribution over time may be measured by calculating the mean intensity values of each color of detected fluorescence for every pixel along the radius or outside edge of the heterogeneous cell mixture, then determining the log₂ ratio of the mean intensity values. See FIG. 6. In various embodiments, the greater distance of the log₂ ratio from the center line indicates the cells distributed and self-organized, while the log₂ ratio that stays close to the mid-center line indicates no self-organization was detected.

Thus in one embodiment, a computer-implemented process comprising calculating the mean intensity values of each color of detected fluorescence for every pixel along the radius or outside edge of the heterogeneous cell mixture, and determining the log2 ratio of the mean intensity values. In another embodiment, a system comprising hardware and software for imaging and detecting cell self-organization, a pixel distribution generator for calculating the mean intensity values and determining the change in cell distribution over time.

Observing and detecting the mechanisms governing self-organization are important in the context of many applications. Table 2 below shows examples of cell types which can be assayed and reasons of interest in studying the conditions that lead to disruption of cell self-organization and tissue formation.

TABLE 2
Examples of Cell Types and Assay Conditions

| Cell types | Interest |
|---|---|
| Normal human mammary | (i) Identification of chemicals, environmental |
| epithelial cells (HMEC). | conditions, epigenetic and genetic modifications that |
| These cell cultures are very | alter maintenance of normal tissue architecture and |
| well characterized and likely | polarity (the first things lost in cancer progression). |
| model other pseudostratified | (ii) Determination of whether or not chemicals for |
| epithelial tissues. | pharmaceutical of industrial use, or environmental |
| | conditions are likely to alter or impair normal |
| | architecture. |
| | (iii) Use of HMEC from women of different ages |
| | will allow exploration of age as a co-factor in i and ii. |
| | (iv) Exploration of basic mechanisms governing self- |
| | organizing behavior. |
| Mixtures of normal and | (i) Identification of chemicals, environmental |
| transformed human mammary | conditions, epigenetic and genetic modifications that |
| epithelial cells (e.g. normal | restore normal tissue architecture. (e.g. transformed |
| myoepithelial cells and | luminal cells or progenitors surrounded by normal |
| transformed luminal cells or | myoepithelial cells could model ductal carcinoma in |
| progenitors.) | situ and one could screen for drugs that correct the |
| | pathological phenotype.) |
| | (ii) Determination of whether chemicals for |
| | pharmaceutical of industrial use, or environmental |
| | conditions, are likely to cause pre-malignant cells to |
| | generate disrupted normal organization |
| Normal cells from any | (i) Identification of chemicals, environmental |
| epithelium | conditions, epigenetic and genetic modifications that |
| | alter maintenance of normal tissue architecture and |
| | polarity (the first things lost in cancer progression). |
| | (ii) Determination of whether or not chemicals for |
| | pharmaceutical of industrial use, or environmental |
| | conditions are likely to alter or impair normal |
| | architecture. |
| Combinations of embryonic | Identification of chemicals, epigenetic and genetic |
| or induced pluripotent state | modifications that steer stem cells into specific |
| stem cells with normal epithelial | microenvironments in order to facilitate their |
| cells. | differentiation. |

In one embodiment, understanding the effects of various test factors upon cell organization is essential is in the area of regenerative tissue maintenance. For example, as MEPs and LEPs are produced anew by mammary progenitor cells in vivo, they must adopt their appropriate place within the tissue, or, alternatively, the progenitors must be able to move to receive instructive microenvironments that direct cell-fate decisions (LaBarge M A, et al. (2009) Human mammary progenitor cell fate decisions are products of interactions with combinatorial microenvironments. Integr Biol 1:70-79, Epub 2008 Nov. 12.). Understanding tissue self-organization mechanisms may help explain how stem cell differentiation and maintenance of tissue architecture in adults are coordinated. Other uses could include identification of strategies for steering embryonic stem cells and induced pluripotent stem cells into specific microenvironmental niches to facilitate their terminal differentiation and incorporation into the target tissue.

Architecture and organization are conserved properties of tissues that distinguish one tissue from another, and loss of normal organization is among the first hallmarks of a number of disease states including but not limited to, cancers, carcinomas and metabolic disorders. Thus, in some embodiments, the assay further provides for methods for diagnosing or prognosing cancer in epithelial tissue, where the determination that epithelial cells in the specific epithelial tissue having an abnormal cellular organization is indicative of aggressive or metastatic cancer.

Future applications of this assay could include screening of chemicals to determine whether they impose, restore, or disrupt normal bilayered organization that is common among pseudostratified and stratified epithelia. This could be useful for pharmaceutical designers trying to identify compounds with minimal effects on normal tissues, or trying to determine whether non-genotoxic compounds that are environmentally derived or are used for industrial/commercial applications may cause changes in normal tissues. This assay system may also be used to screen for compounds that correct aberrant tissue architecture.

### Example 1

### Quantification of Self-Organizing Activity in Different Lineages of Normal Human Mammary Epithelial Cells

We sought to demonstrate that cells possessed lineage-specific intrinsic abilities to self-organize into domains of lineage specificity. Chanson L, Brownfield D, Garbe J C, Kuhn I, Stampfer M R, Bissell M J, LaBarge M A, “Self-organization is a dynamic and lineage-intrinsic property of mammary epithelial cells,” Proc Natl Acad Sci USA. 2011 Feb. 22; 108(8):3264-9, hereby incorporated by reference in its entirety including the supplemental material, for all purposes. We first used a classical self-organization assay to determine whether different lineages of cultured HMEC derived from reduction mammoplasty possessed an intrinsic ability to form bilayered structures. Subpopulations of LEPs and MEPs, defined as CD227⁺/CD10⁻/keratin 19 (K19)⁺/keratin 14 (K14)⁻ and CD227⁻/CD10⁺/K19⁻/K14⁺, respectively (Villadsen R, et al., (2007) Evidence for a stem cell hierarchy in the adult human breast. J Cell Biol 177:87-101), were enriched by FACS from heterogeneous normal finite-lifespan HMEC (Garbe J C et al., (2009) Molecular distinctions between stasis and telomere attrition senescence barriers shown by long-term culture of normal human mammary epithelial cells. Cancer Res 69:7557-7568) at passage 4 or 5 (FIGS. 1 A, A′, and A″). The two lineages were labeled with long-lasting fluorescent membrane dyes of different wavelengths, mixed together, and then were suspended in hanging droplets. The formation of cores of LEPs surrounded by MEPs (FIG. 1B), similar to their organization in vivo, was observed over 48 h. However, the considerable variation in aggregate size, shape, and focal planes precluded a quantitative understanding of the phenomenon.

Therefore, a microwell culture platform was engineered that confined the HMEC mixtures to a 3D cylindrical geometry, which enabled quantification of lineage distributions over time (FIG. 16). Representative optical sections of mixed LEPs and MEPs in microwells, taken at middepth (˜25 μm) at 0 and 48 h, suggest self-organization had occurred, as compared with mixtures of arbitrarily labeled HMEC cultures (FIG. 1D). Time-lapse microscopy from one well demonstrates the dynamic nature of the organizing process (Fig. S1). Heat maps showing the lineage distributions over time suggested that in a majority of microwells MEPs formed a ring surrounding cores of LEPs as early as 24 h, and lasted for at least 48 h (FIG. 1E, Upper). A 1:1 ratio of LEPs to MEPs was determined empirically to provide the most clearly separable distributions, as compared with ratios of 1:2 or 1:3. Using relatively more LEPs than MEPs (e.g., in a ratio of 2:1 or 3:1) was difficult due to the paucity of LEPs. Arbitrarily labeled HMEC cultures, mixed at a 1:1 ratio, showed overlapping distributions of cells that did not resolve into distinct populations (FIG. 1E, Lower). Quantification of the heat maps (Fig. S1) confirmed that a core of LEPs surrounded by MEPs was observable as early as 24 h, and showing as much as a fourfold difference in LEP:MEP ratios at the core versus the periphery by 48 h (P<0.001) (FIG. 1E′, Upper). By contrast there was no difference in ratios at the core and the periphery of the arbitrarily labeled HMEC controls at any time point (FIG. 1E′, Lower). Inflections in the graphs sometimes were observed toward the peripheral regions because of imperfect registration of the well images. Taken together, these results indicate that self-organizing is an innate property of the LEP and MEP HMEC lineages.

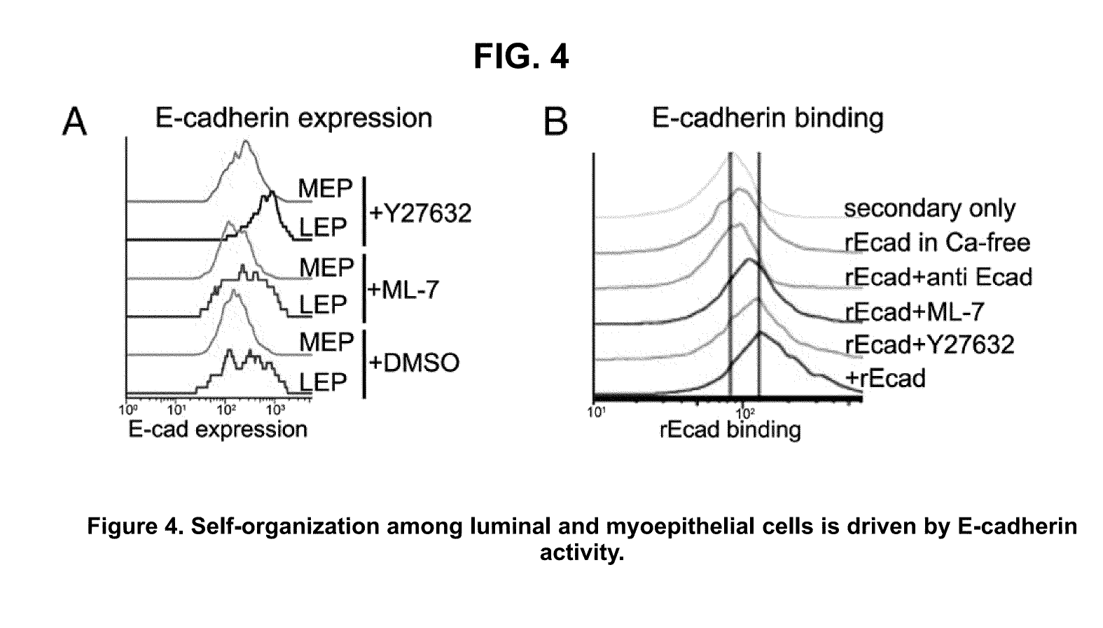

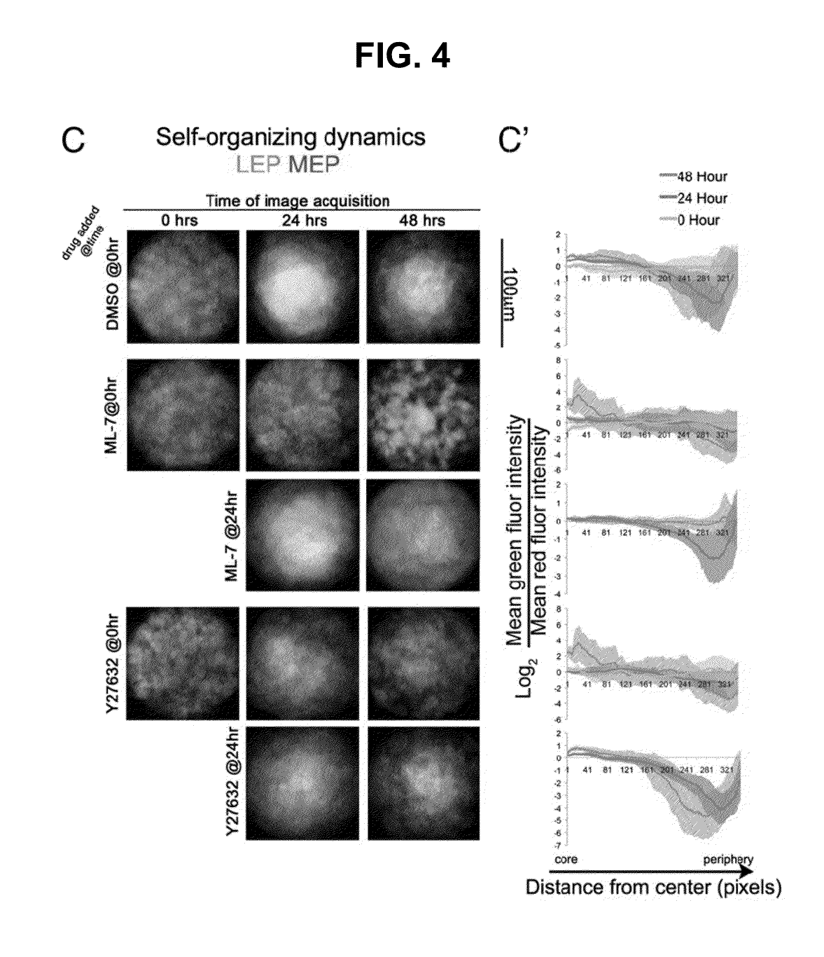

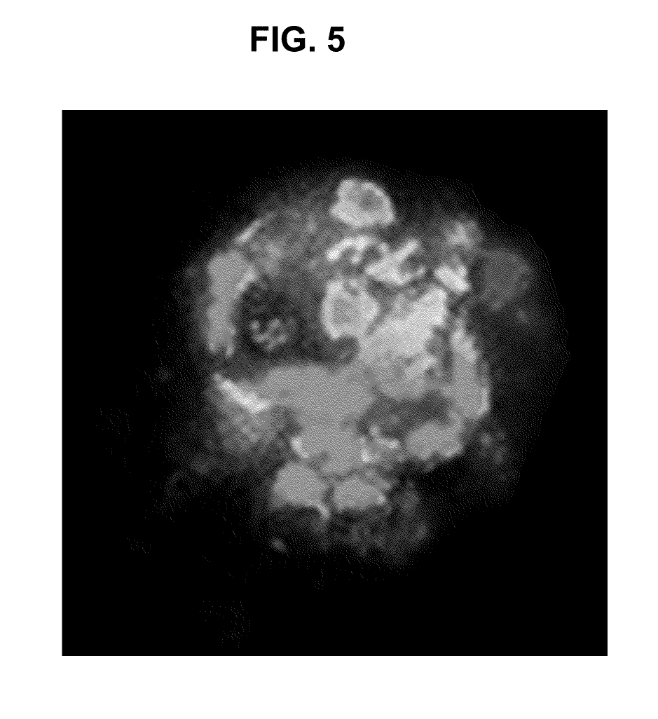

Here we demonstrated that self-organization of mammary epithelial cells is a lineage-specific process that is principally E-cadherin driven; however, P-cadherin also may play a role in organizing the MEP layer. Unaltered normal finite-lifespan HMEC and the microwell assay were used together with recombinant proteins and antibodies that blocked specific adherens junction proteins. The elegant proof-of-principal experiments, which that showed differential levels of cell-cell adhesion molecules can drive self-organizing, were performed using fibroblasts and other immortal cell lines that were engineered to express different levels of adherens junction proteins. It is remarkable, given the undoubted complexity of the LEP and MEP cell surfaces, that E-cadherin plays so central a role in the process of self-organization in those cells. It has been hypothesized that self-organizing is not simply the result of differential levels of cadherin expression or of binding affinities, but rather that adhesion energy and the ability to remodel cell-cell junctions are crucial determinants (Borghi N, James Nelson W, (2009) Intercellular adhesion in morphogenesis: Molecular and biophysical considerations. Curr Top Dev Biol 89:1-32). Dynamic analysis of HMEC in the microwell assay platform in the presence of actomyosin inhibitors provided support for that hypothesis in the context of mammary gland (FIG. 4 C-C′). Elegant time-lapse imaging studies of mouse mammary organoid morphogenesis also revealed that the actomyosin inhibitors Y27632 and ML-7 disrupted the clean bilayered organization, but not to the catastrophic extent observed in the HMEC microwell assay. Because the mouse mammary organoids were developed in vivo, a number of additional cellular interconnectivities crucial for tissue stability may have formed that were absent in our recombined system. Although we focused on cell-cell E-cadherin junctions, other adhesive and physical interactions, such as desmosomal interactions between LEPs and MEPs, undoubtedly are important in maintaining mammary gland organization and bear further dissection. Cell-extracellular matrix (ECM) interactions also will likely affect sorting in vivo. Because the microwell assay uses a nonfouling coating to prevent cell adhesion, the adherens junction proteins may have had a more pronounced effect on self-organizing than they would have had in the presence of ECM. Atomic force microscopy analysis of LEP and MEP on plastic dishes indicated that LEP tended to be softer than MEP. However, a cultured murine epithelial cell line became less stiff in contact with laminin-111, a principal component of basement membrane, than when in contact with plastic (Alcaraz J, et al., (2008) Laminin and biomimetic extracellular elasticity enhance functional differentiation in mammary epithelia. EMBO J 27:2829-2838). Therefore, MEPs in vivo may be less or equally as stiff as LEPs because of their direct contact with basement membrane. Future iterations of the microwell platform will help elucidate more of the factors involved in making stable and organized tissues.

Studying self-organizing behavior of a human epithelium generally is challenging because results cannot be extrapolated easily to in vivo conditions. However, observations of breast cancer pathogenesis suggest the basic mechanisms described here are important for maintaining mammary gland organization. The transcription factor snail controls epithelial-mesenchymal transitions by repressing E-cadherin expression. Nat Cell Biol 2:76-83). E-cadherin expression and localization frequently are misregulated in breast cancers (Zhang X, et al. (2009) Atypical E-cadherin expression in cell clusters overlying focally disrupted mammary myoepithelial cell layers: Implications for tumor cell motility and invasion. Pathol Res Pract 205:375-385; Korkola J E, et al., (2003) Differentiation of lobular versus ductal breast carcinomas by expression microarray analysis. Cancer Res 63:7167-7175; and Moll R, Mitze M, Frixen U H, Birchmeier W (1993) Differential loss of E-cadherin expression in infiltrating ductal and lobular breast carcinomas. Am J Pathol 143:1731-1742), and loss of E-cadherin is a hallmark of the epithelial-to-mesenchymal transition, which is associated with invasive and aggressive breast cancer (Cano A, et al. (2000)

### Levels of E-Cadherin Expression are Lineage Specific

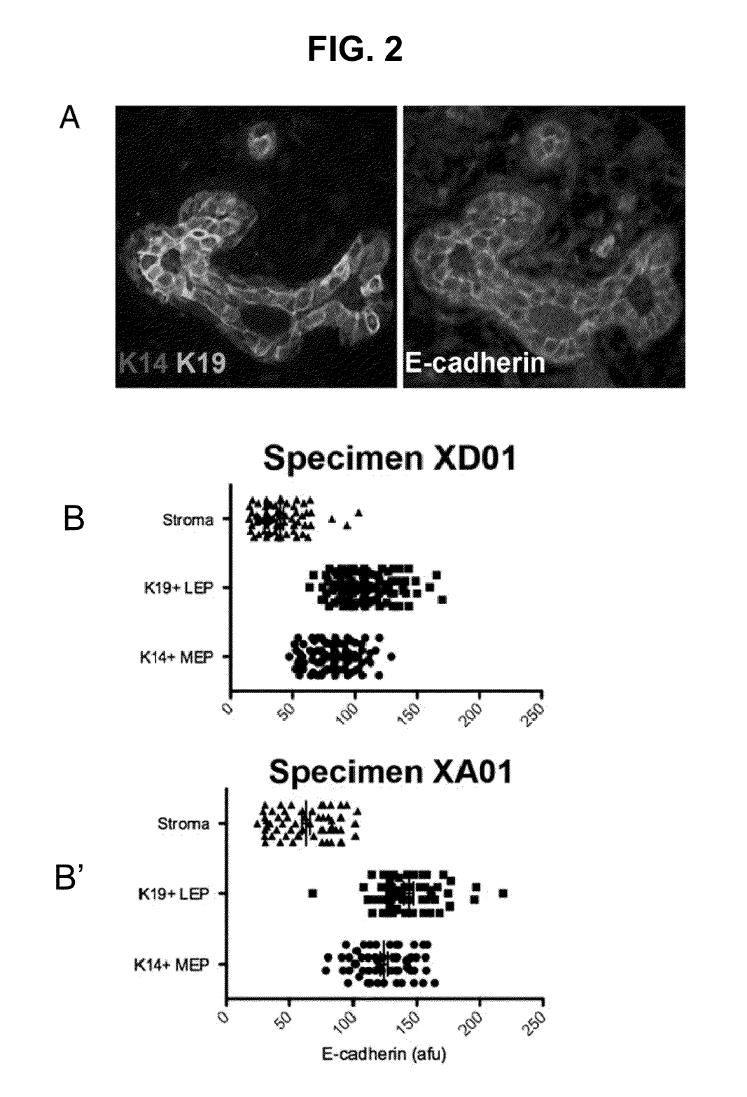

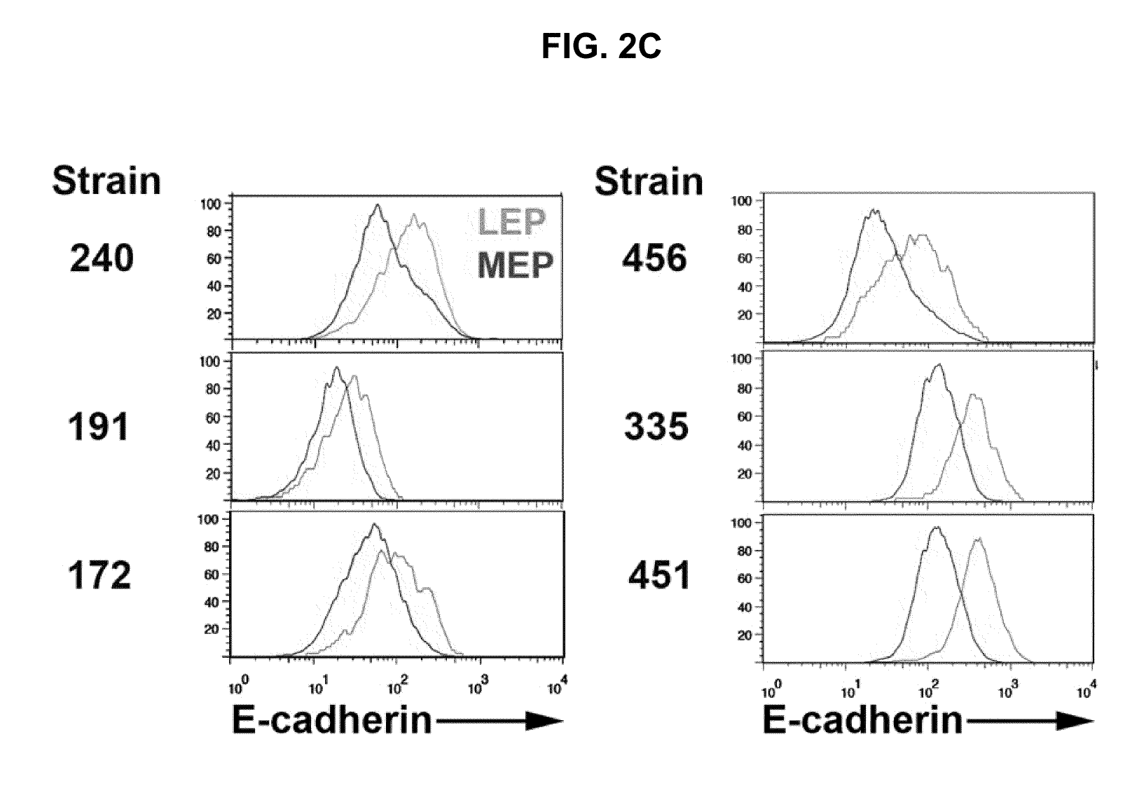

Self-organizing behavior has been ascribed to disparate adhesive properties among the participating cells in embryonic progenitors from the three germ layers, in cancer cell lines, and in fibroblasts engineered to express cell-cell adhesion molecules (the differential adhesion hypothesis, reviewed in Foty R A, et al., (2004) Cadherin-mediated cell-cell adhesion and tissue segregation in relation to malignancy. Int J Dev Biol 48:397-409. Cadherin cell-cell adhesion molecules, particularly E-cadherin, play key roles in tissue morphogenesis during vertebrate gastrulation (Gumbiner B M, (2005) Regulation of cadherin-mediated adhesion in morphogenesis. Nat Rev Mol Cell Biol 6:622-634). Quantification of images of fluorescently immunostained tissue sections of normal mammary gland (FIG. 2A) from two individuals revealed that more E-cadherin protein was present at the borders between two LEPs than at the borders between a LEP and a MEP (P<0.001) (FIG. 2B). Flow cytometry measurements of E-cadherin surface protein levels were made on LEPs and MEPs. In HMEC strains at fourth passage from six individuals, a reproducible pattern was observed, whereby more E-cadherin was detected on LEPs than on MEPs (FIG. 2C). The lineage-specific expression levels of E-cadherin made it an attractive candidate for further testing of the differential adhesion hypothesis as it pertains to self-organization among HMEC.

Functional Identification of Adhesion Molecules that Drive Tissue Self-Organization

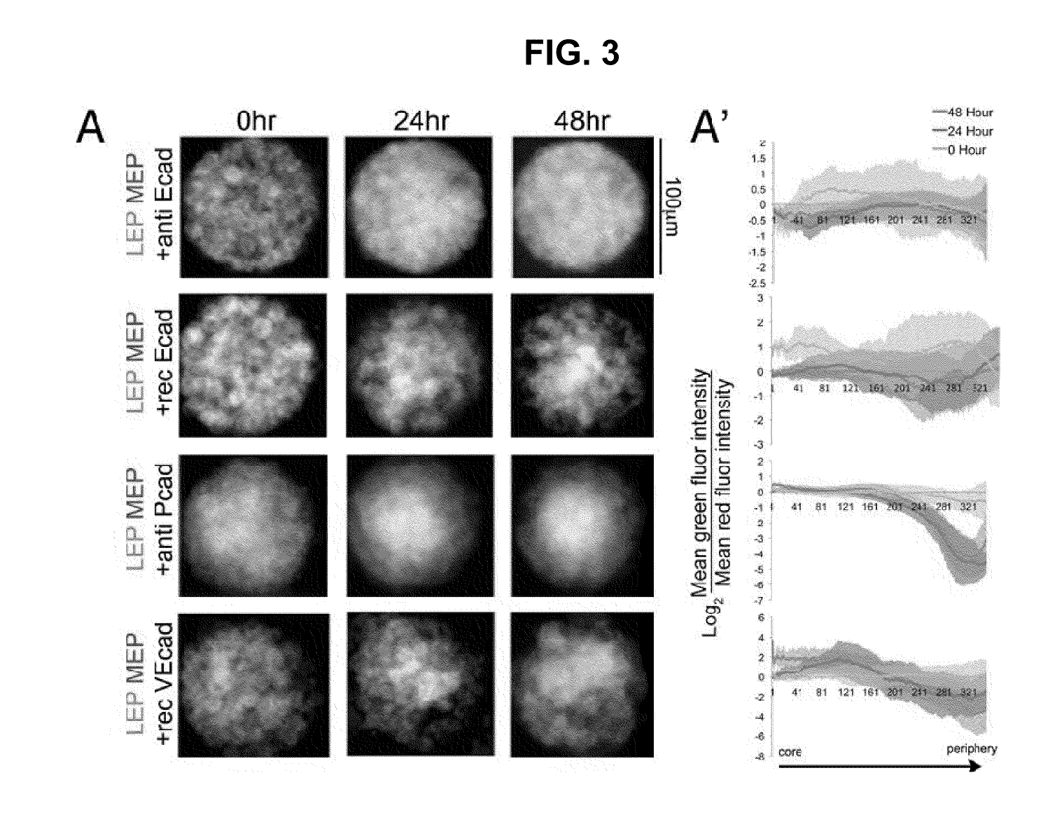

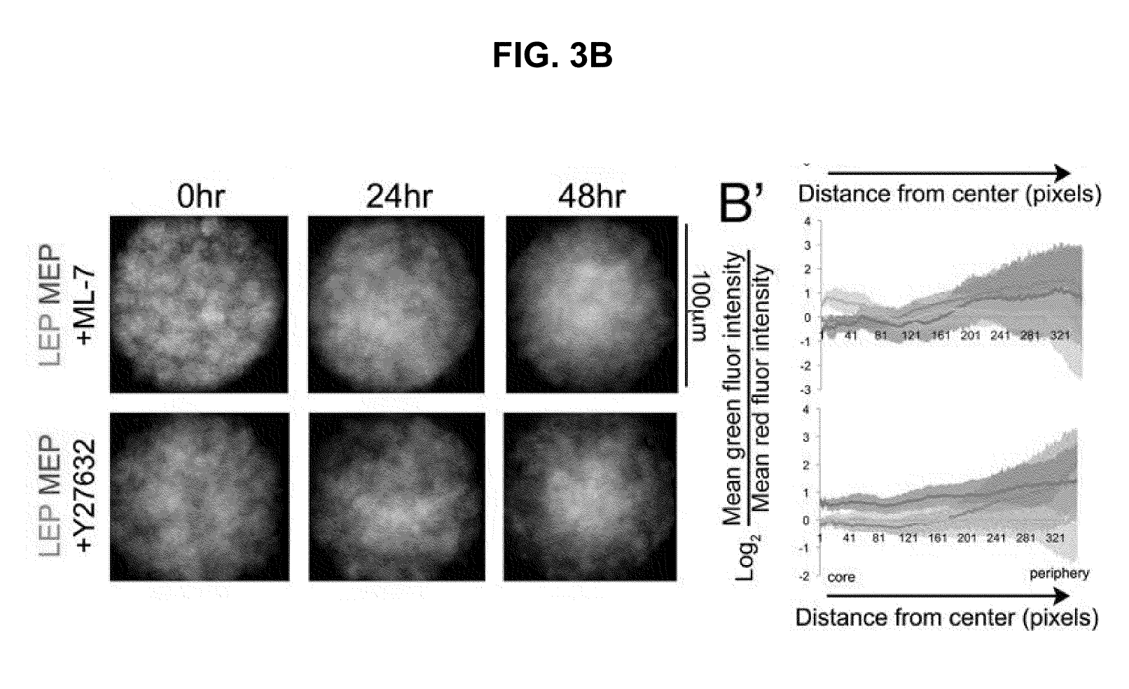

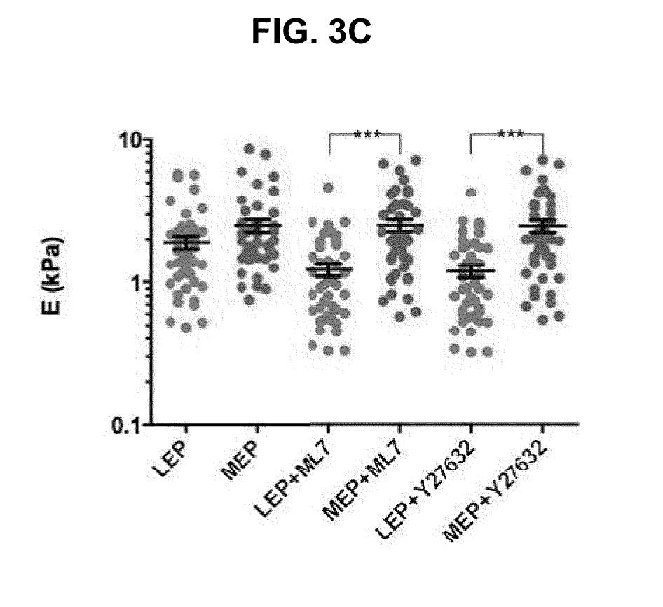

To determine whether cadherins played a functional role in the self-organization of LEPs and MEPs, inhibitors of E-, P-, and VE-cadherin were added to the medium of the microwell assay to antagonize those specific cell-cell interactions. P-cadherin is expressed by MEPs in vivo but not by LEPs (Shimoyama Y, et al., (1989) Cadherin cell-adhesion molecules in human epithelial tissues and carcinomas. Cancer Res 49:2128-2133). VE-cadherin is expressed by endothelial cells but not by epithelial cells (Gumbiner et al., (2005) Regulation of cadherin-mediated adhesion in morphogenesis. Nat Rev Mol Cell Biol 6:622-634) and was used as a control for potential effects of heterotypic cadherin interactions (Shi Q et al., (2008) Biophysical properties of cadherin bonds do not predict cell sorting. J Biol Chem 283:28454-28463). Each of the putative inhibitors was added at the beginning of the experiment and was refreshed every 24 h with medium changes. An antibody that blocked E-cadherin, and recombinant E-cadherin fused to the human IgG-Fc region (recEcad), prevented self-organizing of LEPs and MEPs. Quantification of the heat maps did not reveal differences in LEP:MEP ratios at the core and periphery (FIGS. 3 A and A′). An antibody that blocked P-cadherin did not abolish sorting, because a core enriched for LEPs was surrounded by MEPs (P<0.001) (FIGS. 3 A and A′). However, quantification revealed that there were more MEP at the core [hovering around a ratio of 1:1 (FIG. 3 A′)] than in untreated LEPs and MEPs, which usually showed about a twofold enrichment of LEPs at the core (FIGS. 1 E and E′, Upper). Those data suggest that LEPs organization at the core is unaffected by P-cadherin antibodies, whereas the MEPs were relatively more challenged in their journey to the periphery. Recombinant VE-cadherin IgG-fusion protein (recVEcad) did not prevent organizing (FIGS. 3 A and A′). These data suggested that differential levels of E-cadherin at the surfaces of LEPs and MEPs were the principle drivers of self-organization and thus were the focus of the majority of subsequent studies. However, these data also show that P-cadherin plays a more lineage-restricted role in MEP self-organizing

Self-Organization was Prevented after Negative Modulation of the Actomyosin Network

Previous studies of mammary epithelial morphogenesis have implicated profound roles for the actomyosin regulatory network in normal morphogenesis (Ewald A J et al., (2008) Collective epithelial migration and cell rearrangements drive mammary branching morphogenesis. Dev Cell 14:570-581; Vargo-Gogola T et al., (2006) P190-B Rho GTPase-activating protein overexpression disrupts ductal morphogenesis and induces hyperplastic lesions in the developing mammary gland. Mol Endocrinol 20:1391-1405). We therefore examined the impact on HMEC self-organization of the actomyosin network inhibitors ML-7, a myosin light-chain kinase (MLCK) inhibitor (Makishima M et al, (1991) Induction of differentiation of human leukemia cells by inhibitors of myosin light chain kinase. FEBS Lett 287:175-177), and Y27632, a Rho kinase (ROCK) inhibitor that blocks both ROCK1 and ROCK2 (Davies S P, et al, (2000) Specificity and mechanism of action of some commonly used protein kinase inhibitors. Biochem J 351:95-105). Inhibitors were added at the beginning of the experiment and were refreshed every 24 h with medium changes. Analysis of LEPs and MEPs distributions over 48 h revealed that both inhibitors prevented self-organization; there were no differences in lineage distribution between the core and peripheral regions (FIGS. 3 B and B′). Modulation of the actomyosin network also is known to cause changes in the elasticity and cortical tension of cells. Self-organization studies of germline progenitor cells dissociated from zebra fish embryos suggested that differential actomyosin-dependent cell-cortex tension was a crucial component of self-organization in that system (Krieg M, et al., (2008) Tensile forces govern germ-layer organization in zebrafish. Nat Cell Biol 10:429-436). Stiffer cells organized to the inside and were surrounded by softer cells, and disruption of the stiffness relationship by actomyosin network inhibitors led to deficits in self-organization (Ibid). Using an atomic force microscope, we measured the elasticity of untreated and inhibitor-treated MEPs and LEPs. Although untreated MEPs tended to be stiffer than LEPs, that difference became significant only in the presence of the inhibitors (P<0.001) (FIG. 3C). MEP stiffness was unaffected by ML-7 and by Y27632, but those inhibitors caused softening of LEPs (FIG. 3C). In HMEC the actomyosin network inhibitors increased the magnitude of the difference in elasticity between LEPs and MEPs but did not alter their relative elasticity (i.e., LEPs always were softer than MEPs), suggesting that in this system self-organization was not driven by differential elasticity.

Perturbations of the Actomyosin Network in the Microwell Platform Revealed that Self-Organization is Dynamic and Reversible

How did the actomyosin inhibitors upset the self-organizing mechanism? We investigated whether the actomyosin inhibitors affected expression or binding activities of E-cadherin in HMEC. Addition of ML-7 or Y27632 to the HMEC culture medium did not change lineage-specific differences in E-cadherin expression as measured via flow cytometry; invariably, LEPs expressed more E-cadherin than did MEPs (FIG. 4A). The ability of recEcad simply to bind surfaces of HMEC in suspension also was measured by flow cytometry. Binding of recEcad did not occur in Ca²⁺-free medium or when HMEC were preincubated with an E-cadherin-blocking antibody (FIG. 4B). A 6-h pretreatment with Y27632 or ML-7 did not prevent recEcad binding (FIG. 4B). Therefore, neither differential expression levels of E-cadherin nor its ability simply to bind other E-cadherin molecules at the surface was impacted by ML-7 or Y27632.

Because of the central importance of the cytoskeleton in adherens junction regulation, we considered the possibility that the actomyosin inhibitors modulated the ability of E-cadherin junctions to mature or remodel (Fukata M, et al., (2001) Rho-family GTPases in cadherin-mediated cell-cell adhesion. Nat Rev Mol Cell Biol 2:887-897), thereby impacting HMEC organization. Disruption of MLCK would prevent proper localization of myosin IIA to the E-cadherin junction, disrupting E-cadherin clustering and decreasing homophilic adhesion (Smutny M, et al, (2010) Myosin II isoforms identify distinct functional modules that support integrity of the epithelial zonula adherens. Nat Cell Biol 12:696-702). Conversely, mature adherens junctions were unable to break down and recycle in the presence of Y27632 in HCT116 and MDCK epithelial cell lines (Sahai E et al, (2002) ROCK and Dia have opposing effects on adherens junctions downstream of Rho. Nat Cell Biol 4:408-415). Those reports predicted that ML-7 would ablate already-organized HMEC structures, whereas Y27632 would preserve them. Accordingly, ML-7 or Y27632 was added to mixtures of LEPs and MEPs in the microwell assay, either just after cells were added to wells at the start of the assay (0 h) or after 24 h, when the DMSO controls already started to show signs of organization. In contrast to the experiments shown in FIG. 3B, in which the inhibitors were refreshed every 24 h, in these experiments the inhibitors were added a single time, with the expectation, based on empirical findings, that the inhibitor's activity would begin to weaken by 48 h. This protocol tested the reversibility of the system, because in one condition sorting would be prevented from the beginning and then gradually would be unleashed, and in the second condition sorting would be allowed to get underway before perturbation by the inhibitors after 24 h. Both inhibitors, when added at 0 h, prevented self-organization through the 24-h time point, but as the inhibitors' activity diminished significant, differences in LEP:MEP ratios in the core and at the periphery were observed by 48 h (P<0.01) (FIGS. 4 C and C′). The unique phenotypes of each inhibitor were revealed when they were added after the assay had been underway for 24 h. Before addition of the inhibitors, the LEP were enriched at the core and were surrounded by peripheral MEPs (P<0.001) (FIGS. 4 C and C). When measured at 48 h, addition of ML-7 had obliterated organization, eliminating any difference in the distributions of the lineages (P=ns), whereas Y27632 had preserved the self-organized LEP cores that were encircled by MEPs (P<0.001) (FIGS. 4 C and C′). As a whole, these observations are consistent with the following model: ML-7 prevented adherens junction formation or maturation, and breaking the adherens junctions prevented cells from self-organizing and caused dissolution of already organized structures. Conversely, Y27632 prevented adherens junction recycling, so the cells could not let go of one another to sample the surrounding microenvironment. Thus, both establishing and maintaining organized states are dynamic and reversible processes.

### Materials and Methods

### Cell Culture

HMEC strains were established and maintained according to previously reported methods (J C Garbe et al, (2009) Molecular distinctions between stasis and telomere attrition senescence barriers shown by long-term culture of normal human mammary epithelial cells. Cancer Res 69:7557-7568; Stampfer et al. (1985) Induction of transformation and continuous cell lines from normal human mammary epithelial cells after exposure to benzo[a]pyrene. Proc Natl Acad Sci USA 82:2394-2398), and also described in US Pat. Publication No. 20100022000, all of which are hereby incorporated by reference. Cells were maintained in M87A medium and used for assays at fourth and fifth passages; strain 240L was the only strain used for self-organizing and binding assays.

### Microwell Self-Organization Assay

Micropatterned substrata were made according to Tan et al. (2004) “Simple approach to micropattern cells on common culture substrates by tuning substrate wettability”. Tissue Eng 10:865-872, hereby incorporated by reference. Polydimethylsiloxane (PDMS) microwell arrays were formed by curing prepolymer with base:cure ratio of 10:1 (Sylguard 184) against a prepatterned master. The arrays of wells were peeled away and were cut into 1-cm² pieces that were affixed with a few microliters of uncured PDMS to the bottom of a 24-well plate (Mitek). Plates with microwells were UV oxidized for 7 min (UVO-Cleaner 42; Jelight Co.), blocked with 2 mg/mL BSA (Sigma) for 1 h under vacuum, and rinsed with PBS and M87A. All self-organizing experiments were conducted with HMEC strain 240L. Flow cytometry-sorted HMEC were stained with CM-DiI, SP-DiOC18 (Steinberg M S, (1962) On the mechanism of tissue reconstruction by dissociated cells. I. Population kinetics, differential adhesiveness, and the absence of directed migration. Proc Natl Acad Sci USA 48:1577-1582), or DiIC18(Wei C, et al., (2007) Self-organization and branching morphogenesis of primary salivary epithelial cells. Tissue Eng 13:721-735)-DS (Invitrogen), used at 1:1,000 in PBS for 5 min at 37° C. followed by 15 min at 4° C. Cells were washed extensively with medium after staining Dye-stained HMEC were mixed at a ratio of 1:1 (LEP:MEP) or 1:1 (randomly stained green:red HMEC cultures) and were resuspended in M87A at 1 million cells/mL. Inhibitors were added to the cell suspensions just before HMEC were introduced into the wells and were allowed to load for 30-60 min. Excess cells were washed away with medium; inhibitors then were added to the medium after excess cells were washed away and at every medium change. anti-E-cadherin (100 μg/mL clone HECD-1; Invitrogen); anti-E-cadherin (100 μg/mL clone HECD-1; Invitrogen); anti-P-cadherin (100 μg/mL clone NCC-CAD-299; Abcam); recombinant human (rh) E-cadherin-Fc (recEcad, 100 μg/mL; R&D Systems); rhVE-cadherin (100 μg/mL; R&D Systems); Y27632 (10⁻⁵ M; Calbiochem); or ML-7 at 3×10⁻⁶ M (Calbiochem). HMEC were imaged at 0, 24, or 48 h with a spinning disk confocal microscope (Carl Zeiss). Red and green fluorescence channels in images taken at the ˜25-μm z axis positions of 30 wells from each condition at each time point were binarized using the Threshold function, merged into a Z-stack, and then averaged using ImageJ software (National Institutes of Health). Gray-scaled average images corresponding to LEP and MEP were merged into a single image with red or green look-up tables applied to each average image.

Heat maps were generated. Briefly, heat maps were normalized to the highest intensity value and were used to quantify sorting using the expression log₂(mean green pixel intensity/mean red pixel intensity). A script was written using MATLAB (Mathworks) to plot differential intensity as a function of the distance from the center and to compute the average plot from θ of 0-360° (Fig S2).

### Flow Cytometry Sorting and Assays

HMEC at fourth or fifth passage were trypsinized and resuspended in medium. For enrichment of LEP and MEP images, anti-CD227-FITC (clone HMPV; BD) or anti-CD10-PE (clone HI10a; BioLegend) was added to the medium at 1:50 for 25 min on ice. HMEC then were washed in PBS and sorted on a FACS Vantage DIVA (BD) into their own medium.

E-cadherin expression on LEP and MEP was measured by addition of anti-E-cadherin-A647 (clone 67A4; Biolegend) to the above mixture at 1:50.

To determine effects of inhibitors, Y27632 at 10−5 M (Calbiochem) or ML-7 at 3×10−6 M (Calbiochem) was added to HMEC medium for 6 h at 37° C. (5% CO2) before trypsinization and subsequent measurement of E-cadherin by FACS. To measure the ability of recEcad to bind to HMEC in presence of inhibitors, cells were suspended in their medium in Falcon tubes (to prevent adhesion to a culture surface) in the presence of Y27632 at 10−5M (Calbiochem), ML-7 at 3×10−6M (Calbiochem), or anti-E-cadherin (100 μg/mL; clone HECD-1; Invitrogen), or in calcium-free medium for 6 h at 37° C. (5% CO2). recEcad conjugated to human IgG Fc region was added at 100 μg/mL for 1 h on ice. HMEC then were washed with medium and incubated with anti-human IgG-A633 (1:500; Invitrogen) in their own medium.

### Atomic Force Microscopy Measurements of Stiffness

Once samples were equilibrated to 25° C., cell deformity was measured, and stiffness was calculated as previously described (Alcaraz J, et al., (2008) Laminin and biomimetic extracellular elasticity enhance functional differentiation in mammary epithelia. EMBO J 27:2829-2838.). The resulting data were plotted using Prism(GraphPad Software) (n=45).

### Immunofluorescence Staining.

FACS-sorted HMEC were allowed to adhere to methanol-cleaned coverslips for 2 h. Adherent cells were fixed in methanol:acetone (1:1) at −20° C. for 15 min, blocked with PBS/5% normal goat serum/0.1% Triton X-100, and incubated with anti-keratin 14 (anti-K14) (polyclonal; 1:1,000; Covance) and anti-keratin 19 (anti-K19) (1:20; clone Troma-III; Developmental Studies Hybridoma Bank) overnight at 4° C. Goat anti-rabbit A568 and goat anti-rat A488 secondary antibodies (1:500; Invitrogen), and Hoechst 3342 (1:1,000; Sigma) were added for 2 h at room temp. Cells were imaged with a spinning disk confocal microscope (Carl Zeiss). Sections of formaldehyde-fixed and paraffin-embedded (FFPE) normal human breast tissue (4 μm thick), affixed to slides, were purchased from ProSci (Poway, Calif.). Slides were baked at 55° C. for 1 h to fix the tissue to the slide and to remove much of the paraffin. Complete deparaffinization was done according to the protocol published on the Abcam website. Antigen retrieval was done according to the citrate buffer (pH 6.0)-based protocol published on Abcam website. Slides were not allowed to dry out after deparaffinization and were stored in PBS at 4° C. if not stained immediately. Before reaction with primary antibodies, the slides were blocked for 1 h at room temperature or overnight at 4° C. in normal goat serum (NGS) blocking buffer: 5% NGS, 0.001% azide, 0.1% Triton X-100, and PBS. The unconjugated primary antibody recognizing human cytokeratin 19 was diluted 1:100 in NGS blocking buffer before incubation with the slides in a cold room on a rocker platform overnight, followed by three consecutive 10-min washes in PBS at ambient temperature. Secondary antibodies (goat anti-mouse Alexa Fluor 568) were diluted 1:500 in PBS and incubated with the slides for 1 h at ambient temperature on a slow rocking platform, followed by three 10-min washes in PBS. The conjugated antibodies against K14 (Alexa Fluor 633) and against E-cadherin (Alexa Fluor 488) then were diluted 1:100 in NGS blocking buffer and incubated with the slides overnight in a cold room on a rocker platform, followed by three 10-min washes in PBS. DAPI diluted 1:4,000 in PBS was added to the slides for 5 min; then the slides were rinsed with PBS and destained overnight in PBS in the cold room. The next day slides were overlaid with Fluoromount-G (Southern Biotech) and a #1 coverslip and allowed to dry in the dark at ambient temperature overnight before being sealed with clear nail polish. Imaging was accomplished on a Zeiss 510 spinning disk confocal microscope. For each area of interest, five focal planes of ˜2 μm vertical separation were imaged. Images were processed using Image J software. K14 purified rabbit polyclonal antisera MK14 (AF 64, catalog #PRB-155P; Covance), supplied in PBS, 0.03% thiomersal, was conjugated with Alexa Fluor 633 according to the protocol from Molecular Probes. Briefly, 1 mg Alexa Fluor 633 (Invitrogen/Molecular Probes) was resuspended at 5 mg/mL in acetonitrile and used at 10-fold molar excess to the IgG. Excess Alexa Fluor 633 was aliquoted, rotovapped to dryness, and then was stored in dark at −20° C. K19 Abcam ab7754 (mouse MAb IgG2A) used at 1:100, E-cadherin Ab, Alexa 488 conjugated #3199 from Cell Signaling. The specificity of anti-E-cadherin was determined using recombinant E-cadherin peptide conjugated to the Fc fragment of human IgG (blocking) (catalog #648-EC; R&D Systems) used at 1:5 dilution, equivalent to 50 μg/mL.

### Statistics

E-cadherin images and atomic force microscopy were analyzed using the Kruskal-Wallis test and Dunn's test for multiple comparisons, using a 95% confidence interval. Differences between first and third thirtiles of log₂ (mean green fluorescence/mean red fluorescence) per pixel plotted as a function of distance from the center were analyzed by one-way ANOVA, using Bartlett's test for equal variance and followed by a Tukey's test for multiple comparison using a 99.9% confidence interval. Statistics were computed with Prism (GraphPad Software, Inc.).

The above examples are provided to illustrate the invention but not to limit its scope. Other variants of the invention will be readily apparent to one of ordinary skill in the art and are encompassed by the appended claims. All publications, databases, and patents cited herein are hereby incorporated by reference for all purposes.

What is claimed is:

1. A method for determining loss of organization in a tissue, comprising the steps of:
a. providing a microwell substrate;
b. providing labeled cells from said tissue in the microwells;
c. allowing said cells to self-organize into a specified geometry for a period of time;
d. detecting the changes in localization of said cells over time to self-organize into a specified geometry, whereby little to no changes in localization of said cells over time indicates a loss of organization in said tissue.

2. The method of claim 1, wherein the detecting step d further comprises imaging the labeled cells over time and analyzing the images of said labeled cells.

3. The method of claim 2, wherein analyzing the images of said labeled cells comprised of the steps of (i) calculating the mean intensity values of each label for every pixel along the outside edge of the images of said labeled cells, and (ii) determining the log₂ ratio of the mean intensity values, wherein the greater the distance of the log₂ ratio from the center line indicates changes in the localization of said cells and self-organization is detected, and wherein the log₂ ratio that stays close to the center line indicates no self-organization was detected and a loss of organization in said tissue.

4. The method of claim 1, wherein the label is antibody stain, fluorescence, or a membrane staining fluorescent dye.

5. The method of claim 1, wherein the microwell substrate comprised of a glass or polymer composition.

6. The method of claim 5, wherein the polymer selected from the group consisting of polydimethylsiloxane, polyethyleneglycol, polyacrylamide, polyacrylamide conjugated to collagen 1, and agarose.

7. The method of claim 1, further comprising pre-treating the cells with a test or environmental factor.

8. The method of claim 7, wherein the pre-treatment is a nucleic acid, peptide, protein, drug, small molecule, inhibitor, analyte, chemical, virus, mutation, radiation, temperature change, transformation, or media.

9. The method of claim 1, wherein the cells are a heterogeneous mixture of cell types, wherein each cell type is labeled differently.

10. The method of claim 9, wherein the mixture of cell types is mixtures of normal human epithelial cells, mixtures of normal and transformed human epithelial cells, or combinations of embryonic or induced pluripotent state stem cells with normal epithelial cells.

11. The method of claim 1, wherein the tissue is any pseudostratified epithelial tissue.

12. The method of claim 11, wherein the tissue is breast.

13. A method for screening for loss of organization in a tissue, comprising the steps of:
a. providing a microwell substrate;
b. providing labeled cells from said tissue in the microwells;
c. providing a test factor or treatment to the cells in said microwells, wherein the test factor or treatment is suspected of modulating normal self-organization processes of said cells;
d. allowing said cells to self-organize into a specified geometry for a period of time;
e. detecting the localization of cells over time.

14. The method of claim 13, wherein the detecting step e is compared to a control.

15. The method of claim 1, wherein the detecting step e further comprises (1) imaging the labeled cells over time and (2) analyzing the images of said labeled cells, wherein the analyzing step (2) of the images of said labeled cells comprised of the steps of (i) calculating the mean intensity values of each label for every pixel along the outside edge of the images of said labeled cells, and (ii) determining the log₂ ratio of the mean intensity values, wherein the greater the distance of the log₂ ratio from the center line indicates changes in the localization of said cells and self-organization is detection, and wherein the log₂ ratio that stays close to the center line indicates no self-organization was detected and a loss of organization in said tissue.

16. The method of claim 13, wherein the label is antibody stain, fluorescence, or a membrane staining fluorescent dye.

17. The method of claim 13, wherein the microwell substrate comprised of a glass or polymer composition.

18. The method of claim 17, wherein the polymer selected from the group consisting of polydimethylsiloxane, polyethyleneglycol, polyacrylamide, polyacrylamide conjugated to collagen 1, and agarose.

19. The method of claim 18, wherein the test factor or treatment is a nucleic acid, peptide, protein, drug, small molecule, inhibitor, analyte, chemical, virus, mutation, radiation, temperature change, transformation, or media.

20. The method of claim 13, wherein the cells are a heterogeneous mixture of cell types, wherein each cell type is labeled differently.

21. The method of claim 9, wherein the mixture of cell types is mixtures of normal human epithelial cells, mixtures of normal and transformed human epithelial cells, or combinations of embryonic or induced pluripotent state stem cells with normal epithelial cells.

22. The method of claim 1, wherein the tissue is any pseudostratified epithelial tissue.

23. The method of claim 11, wherein the tissue is breast.

24. A system for carrying out a computer-implemented process comprising the steps of: (a) imaging labeled cells over time and (b) analyzing the images of said labeled cells, wherein the analyzing step (b) of the images of said labeled cells comprised of the steps of (1) calculating the mean intensity values of each label for every pixel along the outside edge of the images of said labeled cells, and (2) determining the log₂ ratio of the mean intensity values, wherein the greater the distance of the log₂ ratio from the center line indicates changes in the localization of said cells and self-organization is detection, and wherein the log₂ ratio that stays close to the center line indicates no self-organization was detected and a loss of organization in said tissue.
